(19) United States
(12) Patent Application Publication
(10) Pub. No.: US 2020/0261670 A1
(43) Pub. Date: Aug. 20, 2020

(54) NASAL FILTER POD

(71) Applicant: Resprana, Inc., New York, NY (US)

(72) Inventors: Sophie Frank, San Francisco, CA (US); Marc-Aurelien Vivant, Brooklyn, NY (US); Micah Steiger, New York, NY (US); Sukanya Goswami, Brooklyn, NY (US); Jai Rathore, Madison, WI (US); Andrea McDonald, New York, NY (US)

(21) Appl. No.: 16/794,856

(22) Filed: Feb. 19, 2020

Related U.S. Application Data
(60) Provisional application No. 62/807,799, filed on Feb. 20, 2019.

(51) Int. Cl. A61M 15/08; B01D 46/54; B01D 46/00; A62B 23/06

(52) U.S. Cl. CPC A61M 15/08; B01D 46/543; B01D 46/0028; B01D 46/0032; A61M 2205/7545; B01D 2279/40; B01D 2279/65; A61M 2205/0205; A62B 23/06

(57) ABSTRACT

A nasal pod is described for dispensing a compound such as asthma medication to air inhaled through the nose.

### CLAIM OF PRIORITY

This application claims priority under 35 U.S.C. § 119 to U.S. provisional application No. 62/807,799 filed on Feb. 20, 2019 in the United States Patent and Trademark Office, the entire disclosure of which is incorporated herein by reference.

### FIELD OF THE EMBODIMENTS

This invention relates to a 3-dimensional nasal filter pod for capturing particles present in air inhaled through the nose.

### BACKGROUND OF THE EMBODIMENTS

The prevalence of chronic inflammation disorders of the lungs is on the increase. For example, in the U.S. alone, about 1 in 12 people or about 25 million suffer from asthma, of which 6.3 million are children. Indeed, asthma is the third-ranking cause of hospitalizations for children under the age of 15. It is now well established that exposure to airborne particles such as animal dander, dust mites, cockroach particles, pollen, tobacco smoke, air pollution, and chemical irritants can trigger an asthma episode or attack resulting in coughing, wheezing, chest tightness, and difficult breathing. Both long-term and short-term exposure can cause health problems such as reduced lung function and more asthma attacks. In the most severe cases, asthma causes the death of more than 3,600 Americans each year.

There is an on-going urgent need for devices and methods that reduce the incidence of or exasperation of chronic respiratory inflammatory disorders.

Examples of related art are summarized below.

U.S. Pat. No. 2,057,397 discloses a nasal inhaler consisting of a cone or cup with an entry and exit and a filler of absorbent material such as cotton or wool inserted into the shell

U.S. Pat. No. 5,568,808 discloses a disposable nose filter to be inserted in a nostril, including a flexible housing, a filter component and a flutter valve. The nose filter is adapted to be easily inserted and removed from the nostril. The flutter valve forms a seal with the lower exterior portion of the nostril, thus, forcing air through the filter and preventing air from passing between the housing and the inner walls of the nostril.

U.S. Pat. No. 7,354,467 discloses a filtering assembly for nasal cavities including a connector with two rings connected by a link, and two filtering members securely connected to the two rings. The two filtering members are inserted into the wearer's nasal cavities so as to effectively filter foreign objects from entering to the wearer's lungs.

U.S. Pat. No. 7,918,225 discloses a nasal air filtration device comprising a pair of concave-convex filters, and a support structure incorporating a pair of generally annular bases for supporting the filters, and a bridge that couples the bases, to maintain them in a desired spaced-apart relation and to determine a desired angular relationship between the bases and between the associated filters.

U.S. Pat. No. 8,479,735 discloses a nasal cavity filter having a substantially cylindrical supporting wall which adheres to the walls of a nasal cavity, and an inner surface of which defines a cavity for the passage of air inhaled and exhaled by the user. The cavity houses a number of fins, which generate turbulence in the air flowing through, and the surface of which impacted by the air retains particles present in the air.

The published U.S. Patent Application 2018/0104518 discloses a nasal cavity insertion type mask for filtering various kinds of fine dust contained in external air inhaled through the nose.

The published U.S. Patent Application 2017/0318874 discloses methods for the delivery of beneficial respirable compounds such as moisture, anti-inflammation compounds, anti-irritants, fragrances, aromatherapy compounds, and low dose medications slowly and continuously over time using a mask containing a thickened aqueous gel media delivery system combined with, supported by, and coating solid bodies inside the filter.

The published U.S. Patent Application 2009/0229608 discloses an inhaler comprising a housing to receive an elongate strip of blisters each containing a dose of medicament and being sequentially movable into alignment with means for breaching a blister to enable a user to inhale said dose contained therein.

U.S. Pat. No. 6,012,455 discloses a nasal air delivery apparatus configured to deliver breathable air under continuous positive air pressure to a person who requires such treatment. The apparatus includes a dentally stabilized platform and a nose mask, nose piece or nasal tubes resiliently mounted on the platform and urged toward nasal engagement and rhinal seal. In the case of configurations including nose masks and nose pieces, the force applied from the dentally stabilized platform is through a resilient connection. Various nose masks, nose piece and nostril engaging air tubes carrying nasal pads are disclosed. In each case, the resilient mounting on the dentally stabilized platform provides the necessary air seal. Air which is enhanced by medication or added oxygen may be supplied by the apparatus.

U.S. Pat. No. 878,223 discloses an inhaler designed to be worn continuously, the inhaler itself being inserted in the nostrils of the wearer, where it is retained in position. When in use, the cups or capsules are filled with absorbent cotton, bits of sponge, or any absorbent material, upon which suitable volatile medicines may be dropped until the absorbent material has become saturated.

The International PCT patent application WO2011/041921 discloses a nose mask having a plurality of functions, for example: to filter pollen and bacteria and the like in the air filtering function of particles, volatile medicine for treating function of release, and such as the end of the water-proof function.

The International PCT patent application WO 2005/035066 discloses a pair of mask bodies; accepting spaces; connection parts and filter devices, arranged in the nose mask bodies' accepting space that confer sterilization and bacteriostasis effects.

U.S. Pat. No. 6,792,945 discloses an inhalation device for use with a medicament pack in which at least one container for medicament in powder form is defined between two sheets peelably secured to one another.

U.S. Pat. No. 3,698,390 discloses a medicament dispensing apparatus which includes a first housing and a nozzle of the convergent-divergent type having an inlet and outlet adjacent the opposite ends of a nozzle chamber. A reservoir for liquid medicament is located outside the nozzle chamber adjacent the throat of the nozzle, and a metering and dispensing valve is positioned adjacent the throat for injecting a measured amount of medicament from the reservoir into the nozzle. Upon inhaling at the outlet of the nozzle the medicament injected into the nozzle is drawn into the mouth of the patient.

None of the art described above addresses all of the issues that the present invention does.

For example, none of the references disclose a device comprising a porous continuous membrane disposed over a support structure defining a three-dimensional hollow shape configured to be inserted into a user's nostril and house a filler containing an inhalable compound.

### SUMMARY OF THE EMBODIMENTS

In a first aspect, a device for dispensing a compound to air inhaled through a user's nose is disclosed that comprises a porous membrane disposed over a support structure defining a three-dimensional hollow shape containing a filler material imbibed with a volatile compound, wherein the three-dimensional shape is configured to be inserted into a user's nostril.

In a second aspect, a device for dispensing a compound to air inhaled through a user's nose is disclosed that consists of a porous membrane disposed over a support structure defining a three-dimensional hollow shape containing a filler material imbibed with a volatile compound, wherein the three-dimensional shape is configured to be inserted into a user's nostril.

In certain embodiments of the first or second aspect, the membrane is continuous.

In certain embodiments of the first or second aspect, the device's membrane comprises pores having a diameter of about 50-500 microns.

In certain embodiments of the first or second aspect, the device's membrane comprises pores having a diameter of about 1-25 microns.

In certain embodiments of the first or second aspect, the membrane comprises pores having a diameter of about 1-10 microns.

In certain embodiments of the first or second aspect, the membrane comprises pores having a diameter of about 1 micron.

In certain embodiments of the first or second aspect, the device's membrane comprises pores having a diameter of about 50-300 nanometers.

In certain embodiments of the first or second aspect, the device's membrane comprises pores having a diameter of about a 0.1-5 millimeters.

In certain embodiments of the first or second aspect, the inhaled air comprises allergens, dust particles, viruses, pollution particles and/or airborne particles, for example, resulting of combustion such as the ones resulting from forest fires. In certain embodiments of the first or second aspect, the support structure defines a cylindrical shape.

In certain embodiments of the first or second aspect, the membrane comprises natural or synthetic fibers. In certain embodiments, the synthetic fibers may comprise at least one of polyethylene terephthalate (PET), polyethersulfone (PES), polyvinylidene (PVDF), polytetrafluoroethylene (PTFE, Teflon), polyamide (nylon), carbon fibers, activated carbon, cellulose acetate, cellulose nitrate (collodion), mixed-cellulose esters (MCE), i.e., a mixture of cellulose nitrate/acetate fibers, polycarbonate, polypropylene (PP), polyacrylonitrile (PAN), polymethyl methacrylate (PMMA), polyvinyl alcohol (PVA), polyurethane (PU) or polyvinylchloride (PVC).

In certain embodiments of the first or second aspect, the membrane has electrostatic properties.

In certain embodiments of the first or second aspect, the membrane is fabricated by electrospin blowing, fiber weaving, foam casting, injection molding, compression molding or vacuum forming.

In certain embodiments of the first or second aspect, the filler material is made of woven mesh fabric, foam, non-woven mesh plastic or fabric, a sponge, cotton wool or synthetic material.

In certain embodiments of the first or second aspect, the volatile compound comprises an extract, oil or medicament.

In certain embodiments, the medicament treats a chronic respiratory disease such as asthma.

In a third aspect, a method for dispensing a compound to air inhaled through a user's nose is disclosed comprising inserting the device of any one of the preceding embodiments into each of a user's nostril.

### BRIEF DESCRIPTION OF THE DRAWINGS

FIGS. 1A and 1B show exemplary perspective views of certain embodiments of a 3-dimensional nasal filter pod.

### DESCRIPTION OF THE PREFERRED EMBODIMENTS

The preferred embodiments of the present invention will now be described with reference to the drawings. Identical elements in the various figures are identified with the same reference numerals.

As used herein, the singular forms “a,” “an,” and “the,” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

As used herein, the terms “treat,” “treatment” or “treating” refer to therapeutic treatments, wherein the object is to reverse, alleviate, ameliorate, inhibit, slow down or stop the progression or severity of a disorder, e.g. a respiratory disorder. The term “treating” includes reducing or alleviating at least one adverse effect or symptom of a condition, disease or disorder associated with a disorder, e.g. a respiratory disorder. Treatment is generally “effective” if one or more symptoms or clinical markers are reduced. Alternatively, treatment is “effective” if the progression of the disorder is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” also includes providing relief from the symptoms or side-effects of a disorder, e.g. a respiratory disorder (including palliative treatment).

In certain embodiments, the terms “treat,” “treatment” or “treating” refer to therapeutic treatments, wherein the object is to reverse, alleviate, ameliorate, inhibit, slow down or stop the progression or severity of a disorder that is not a respiratory disorder. The term “treating” includes reducing or alleviating at least one adverse effect or symptom of a condition, disease or disorder associated with a disorder that is not a respiratory disorder. For example, the filler material may comprise a medicament for the treatment of a disorder that is not a respiratory disorder.

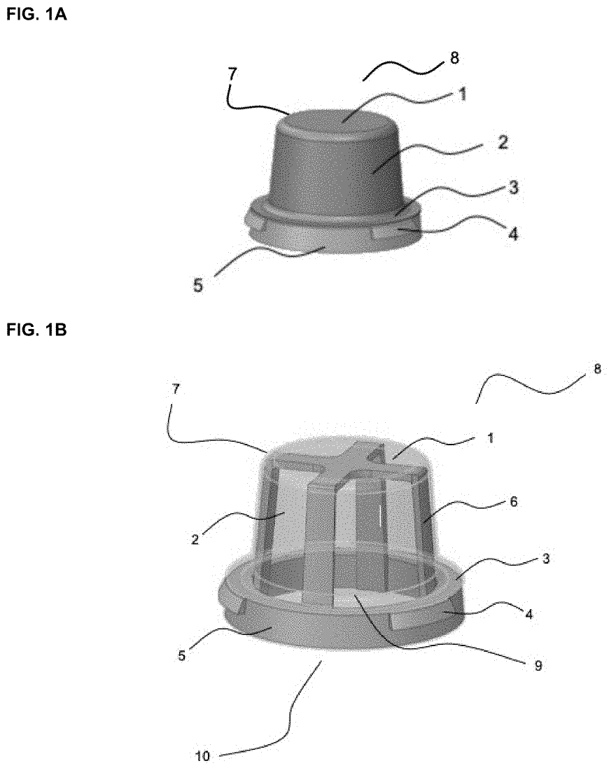

FIG. 1A depicts an exemplary perspective view of a 3-dimensional nasal filter pod 8 having a top 1, side 2, bottom entrance 10 and an interior cavity 9. The porous membrane 7 can have a flange 3 that attaches to a circular support base 5. Protrusions 4 at the periphery of the circular support base 5 help to keep the nasal filter pod in place after insertion into the nasal cavity.

FIG. 1B shows an exemplary perspective view of a 3-dimensional nasal filter pod 8 showing the support structure 6 beneath the porous membrane 7.

In certain embodiments, attachment of the porous membrane 7 to the support base 5 may include, but is not limited to, press fit, slip fit, adhesive, threading, clamps, double-sided tape, heat-seal, high-frequency welding or magnetic attachment.

In certain embodiments, the porous membrane 7 can be manufactured with an electro spin blowing manufacturing process to form a flat 2D membrane that can be molded into a 3-dimensional structure.

In certain embodiments, the porous membrane 7 can be manufactured by a process including, but not limited to fiber weaving, foam casting, injection molding, compression molding or vacuum forming.

In certain embodiments, the porous membrane 7 can be made of natural and/or synthetic fibers. In certain embodiments, the synthetic fibers may include, but is not limited to, at least one of polyethylene terephthalate (PET), polyethersulfone (PES), polyvinylidene (PVDF), polytetrafluoroethylene (PTFE, Teflon), mixed-cellulose esters (MCE), polyimide (nylon), natural fibers, carbon fibers, or activated carbon, cellulose acetate, cellulose nitrate (collodion), mixed-cellulose esters (MCE, i.e., a mixture of cellulose nitrate/acetate fibers), polycarbonate, polypropylene (PP), polyacrylonitrile (PAN), polymethyl methacrylate (PMMA), polyvinyl alcohol (PVA), polyurethane (PU) and polyvinylchloride (PVC). In certain embodiments, the porous membrane 7 may comprise chitosan, collagen, polylactic acid (PLA) and/or Poly(lactic-co-glycolic acid) (PLGA). In certain embodiments, the porous membrane 7 can include activated carbon conferring electrostatic charge to the membrane.

In certain embodiments, the porous membrane 7 can include silver ions and silver nanoparticles to increase the antimicrobial effect of the overall filtration against bacteria and viruses.

In certain embodiments, the inhaled air comprises allergens, dust particles, viruses, pollution particles such as the ones classified under the particulate matter, pm2.5 denomination, and/or airborne particles resulting of combustion such as the ones resulting from forest fires.

In certain embodiments, the porous membrane 7 can have a 3-dimensional (3D) structure, in that it is not limited to a single shape that is cut-out, formed or created as part of or from a two (2) dimensional substrate, such as sheet of paper or a sheet of foam.

In certain embodiments, the porous membrane 7 can have a 3-dimensional shape that creates a maximum surface area for air to flow through, thus decreasing the pressure drop between the air pressure at the entrance of the nasal filter 10 and the pressure at the top 1 of the nasal filter pod 8.

In certain embodiments, the 3-dimensional shape defines a hollow cavity 9.

In certain embodiments, the structural integrity of the nasal filter pod 8 can be re-enforced by a support structure 6 on which the porous membrane 7 can be seated or inserted, stuffed, trapped, encapsulated, merged, woven, or other any means of attachment.

In certain embodiments, methods of attaching the porous membrane 7 to the support structure 6 and support base 5 include, but are not limited to, gluing, bonding, heat staking, melting, friction welding, vibration welding, sonic welding or insert molding (also referred to as overmolding).

In certain embodiment, the porous membrane 7 has pores with an average diameter of about 1, 2, 3, 4, 5, 6, 7, 8, 9, 10, 15, 20 or 25 microns.

In certain embodiment, the porous membrane 7 has pores with an average diameter ranging from 50 to 300 nm.

In certain embodiment, the porous membrane 7 has pores with an average diameter ranging 0.1 to 5 mm.

n certain embodiment, the porous membrane 7 can have a thickness of about 0.1, .0.2, 0.3, 0.4, 0.5, 0.6, 0.7, 0.8, 0.9, 1, 2, 3, 4, 5, 6, 7, 8, 9, or 10 mm.

In certain embodiments, the porous membrane 7 is continuous, e.g., made from a single piece of material.

In certain embodiments, the hollow cavity 9 of the nasal filter pod 8 may contain a porous filler material.

In certain embodiments, the porous filler material can be made of, for example, a porous woven mesh fabric, foam, non-woven mesh plastic or fabric, a sponge, cotton wool and/or synthetic material.

In certain embodiments, the porous filler material can contain a volatile compound such as an oil extract, vapor, scented powder or liquid and/or medicament that is slowly released into the air inhaled through the user's nostril.

In certain embodiments, the porous filler material can be imbibed with a powdered or liquid medication, for example, for the treatment of a chronic respiratory illness such as asthma.

In certain embodiments, the porous filler material containing a medicament may be transferable to devices such as masks or air-purifier like products.

Although this invention has been described with a certain degree of particularity, it is to be understood that the present disclosure has been made only by way of illustration and that numerous changes in the details of construction and arrangement of parts may be resorted to without departing from the spirit and the scope of the invention.

1. A device for dispensing a compound to air inhaled through a user's nose, said device comprising a porous membrane disposed over a support structure defining a three-dimensional hollow shape containing a filler material imbibed with a volatile compound, wherein the three-dimensional shape is configured to be inserted into a user's nostril.

2. The device of claim 1, further comprising a porous membrane disposed over a support structure defining a three-dimensional hollow shape containing a filler material imbibed with a volatile compound, wherein the three-dimensional shape is configured to be inserted into a user's nostril.

3. The device of claim 1, wherein the membrane is continuous.

4. The device of claim 1, wherein the membrane comprises pores having a diameter of about 1-25 microns.

5. The device of claim 1, wherein the membrane comprises pores having a diameter of about 50-500 microns.

6. The device of claim 1, wherein the membrane comprises pores having a diameter of about 50-300 nanometers.

7. The device of claim 1, wherein the membrane comprises pores having a diameter of about 0.1-5 mm.

8. The device of claim 1, wherein the inhaled air comprises allergens, dust particles, viruses, pollution particles and/or airborne particles resulting from a fire.

9. The device of claim 1, wherein the support structure defines a cylindrical shape.

10. The device of claim 1, wherein the membrane comprises natural fibers and/or synthetic fibers, said synthetic fibers comprising at least one of polyethylene terephthalate (PET), polyethersulfone (PES), polyvinylidene (PVDF), polytetrafluoroethylene (PTFE, Teflon), polyamide (nylon), carbon fibers, activated carbon, cellulose acetate, cellulose nitrate (collodion), mixed-cellulose esters (MCE), i.e., a mixture of cellulose nitrate/acetate fibers, polycarbonate, polypropylene (PP), polyacrylonitrile (PAN), polymethyl methacrylate (PMMA), polyvinyl alcohol (PVA), polyurethane (PU) and polyvinylchloride (PVC).

11. The device of claim 1 wherein the membrane comprises silver ions and/or silver nanoparticles to increase an antimicrobial effect against bacteria and viruses.

12. The device of claim 1, wherein the membrane has electrostatic properties.

13. The device of claim 1, wherein the membrane is fabricated by electrospin blowing, fiber weaving, foam casting, injection molding, compression molding or vacuum forming.

14. The device of claim 1, wherein the filler material is made of woven mesh fabric, foam, non-woven mesh plastic or fabric, a sponge, cotton wool or synthetic material.

15. The device of claim 1, wherein the volatile compound comprises an extract, oil or medicament.

16. The device of claim 1, wherein the medicament treats a chronic respiratory disease.

17. The device of claim 1, wherein the chronic respiratory disease is asthma.

18. A method for dispensing a compound to air inhaled through a user's nose comprising' inserting the device of claim 1 into each of a user's nostril.
